(19) United States
(12) Patent Application Publication
(10) Pub. No.: US 2005/0171461 A1
(43) Pub. Date: Aug. 4, 2005

(54) Walking brace

(76) Inventor: Erez Pick, Roslyn Heights, NY (US)

Correspondence Address: JONES DAY, 77 WEST WACKER, CHICAGO, IL, 60601-1692, US

(21) Appl. No.: 10/769,587

(22) Filed: Jan. 30, 2004

(51) Int. Cl. A61F 5/00

(52) U.S. Cl. 602/27

(57) ABSTRACT

A walking brace for immobilizing and/or protecting the lower leg of a patient comprises a leg portion adapted to fit substantially around the lower leg of a patient and a sole portion that fits beneath the patient's foot. The sole portion is provided with a dual layer of resilient shock absorbing material disposed along its bottom surface. The dual layer comprises an upper layer of a relatively softer material and a lower layer of a relatively more durable material. The dual layer can provide good shock absorbency but can be made thinner to provide a lower profile. In another aspect of the invention, the walking brace is provided with one or more inflatable air cells to provide adjustable therapeutic pressure to the leg and the leg portion of the brace is in the form of a rigid shell provided with a protruding region for receiving the air cell outlet associated with each air cell such that the air cell outlet is directed toward the front of the walking brace for easy access by the user. In yet another aspect of the invention, the rigid shell member of the leg portion is provided with one or more longitudinal slits that allow expansion of the leg portion to accommodate an enlarged lower leg portion of a patient. The expanded leg portion can accommodate users with enlarged lower limbs, such as those suffering from severe edema.

### FIELD OF THE INVENTION

The present invention relates generally to orthopedic devices, and more particularly, to those orthopedic devices known variously as casts, splints, braces, etc. which are especially adapted for immobilizing and/or protecting injured limbs or other parts of the anatomy.

### DESCRIPTION OF THE RELATED ART

In the management of certain injuries to the lower extremities such as fractures of the tibia and fibula, malleolar fractures, or severe ankle sprains, it is common to immobilize the lower extremity completely by use of the well-known molded plastic or resin cast. Once the injured extremity has become stable, however, it has been found that recovery may be effected more rapidly by gradually and progressively permitting the extremity to bear weight and undergo other permitted exercises.

For example, an orthopedic brace, such as that disclosed in U.S. Pat. No. 3,955,565, which is assigned to the assignee herein and incorporated herein by reference in its entirety may be used. This brace features one or more rigid outer shell members having associated therewith an inflatable liner or air cell for engaging a body part or limb. Commercial embodiments of the brace incorporating the invention disclosed in this prior patent are adapted to be fixed about the lower leg and typically comprise a rear outer shell member, a front outer shell member, and air cells disposed within the liner of the shell members. Strap fastening means maintain the shell members in engagement with confronting portions of the lower leg whereby each air cell serves as a firm supporting cushion of pressurized air between the irregular contours of the lower leg and the member sidewalls.

This brace construction is capable of stabilizing the ankle and leg while allowing the wearer to walk. Thus, ambulatory functionality and permitted exercises are feasible thereby encouraging more rapid recovery from various injuries to the lower extremity than otherwise would be possible. Moreover, studies have indicated that a pressurized brace yields a stronger healed fracture than a conventional cast. Dale, P. A. et al., “A New Concept in Fracture Immobilization,” Clinical Orthopedics and Related Research, 264-269 (1993).

An improved version of an orthopedic walking brace is disclosed in U.S. Pat. No. 5,577,998, also assigned to the assignee herein and incorporated herein by reference in its entirety. This walking brace includes a passive reinflation means disposed within the air cells so that no external equipment or oral inflation tube for reinflation of the air cells is required.

Various types of braces are known for this purpose. All such braces include a leg portion that fits on to the patient's lower leg, and a sole portion that fits beneath the patient's foot. The leg portion can be, for example, a pair of opposing splints connected by foam, although a rigid plastic shell leg portion is preferred. In all such cases, however, the sole portion will be a rigid material to properly support and position the foot with respect to the leg during activity.

Another improved version of an orthopedic walking brace is disclosed in U.S. Pat. No. 6,027,458, also assigned to the assignee herein and incorporated herein by reference in its entirety. The walking brace disclosed therein has enhanced shock absorbency at the patient's heel. In particular, a layer of shock absorbing material disposed along the bottom of the walking brace has a heel portion that extends upwardly through an opening in the sole portion of the brace to engage the patient's heel. Some patients experience difficulty or discomfort when using a walking brace because the foot of the injured leg is raised relative to the foot of the uninjured leg such that the patient's legs are effectively two different lengths, thereby making walking even more difficult. It is thus desirable to provide a walking brace wherein the amount by which the injured foot is raised relative to the uninjured foot is minimal, yet while providing protection and shock absorbency at the sole portion.

Another issue relating to patient comfort and compliance is the ease of access to adjustable air cells in the device. It would be desirable to provide a walking brace in which the air cells can be readily accessed by the user to adjust the therapeutic air pressure as needed. Yet another issue relating to patient comfort is the fit of the upper end of the leg portion around the patient's ankle and calf. Some patients experience swelling in their lower legs and angles. This can be due to edema, or to other pathologies. It would be desirable to provide a walking brace in which the width of the leg portion can be expanded to accommodate those patients with swollen limbs.

It is thus one object of the invention to provide a walking brace with improved user comfort that provides less of a rise of the foot of the injured limb relative to the foot of the uninjured limb.

It is another object of the invention to provide a walking brace with improved user comfort by providing inflatable air cells that are readily accessible to the user for air pressure adjustment.

It is still another object of the invention to provide a walking brace with improved user comfort in which the rigid shell can expand to accommodate swollen limbs such as from edema and the like.

Other objects, advantages, and novel features of the invention will be readily apparent from the description of the invention and the accompanying drawings.

### SUMMARY OF THE INVENTION

The present invention comprises a walking brace for immobilizing and/or protecting the lower leg of a patient, the brace comprising a leg portion adapted to fit substantially around the lower leg of a patient and a sole portion that fits beneath the patient's foot. The sole portion is provided with a dual layer of resilient shock absorbing material disposed along its bottom surface. The dual layer comprises an upper layer of a relatively softer material and a lower layer of a relatively more durable material. The sole portion of the leg brace is provided with an opening in the area of the user's heel such that the patient's heel contacts the upper layer of the dual layer of resilient shock absorbing material when the patient walks. The dual layer can provide good shock absorbency but can be made thinner to provide a lower profile. The foot of the injured limb will not be raised as height with respect to the foot of the uninjured limb as in prior art braces, for greater patient comfort during walking.

In yet another aspect of the invention, the walking brace is provided with one or more inflatable air cells to provide adjustable therapeutic pressure to the leg. The leg portion of the brace is in the form of a rigid shell provided with a protruding region for receiving the air cell outlet associated with each air cell in such a manner that the air cell outlet is directed toward the front of the walking brace for easy access by the user. Thus, the user does not have to reach behind the leg in order to adjust the air pressure as in prior art designs.

In yet another aspect of the invention, the rigid shell member of the leg portion is provided with one or more longitudinal slits that allow expansion of the leg portion to accommodate an enlarged lower leg portion of a patient. In some embodiments, these longitudinal can have frangible septa at one or both ends thereof. These septa can be cut or otherwise broken to allow the width of the leg portion to expand. The expanded leg portion can accommodate users with enlarged lower limbs, such as those suffering from severe edema.

### DESCRIPTION OF THE DRAWINGS

FIG. 1 is a perspective view of a preferred embodiment of a walking brace of the instant invention;

FIG. 2 is a front view of the front view of the walking brace of FIG. 1 with the walking brace in the closed condition;

FIG. 3 is a front view of the walking brace in the open condition and with the front piece of the exterior shell removed;

FIG. 4 is a side view of the walking brace of FIG. 1 with the foam pieces unfolded and the front piece removed;

FIG. 5 is a perspective view of the rear portion of the exterior shell of the walking brace;

FIG. 6 is a cross-sectional view taken through line 6-6 of FIG. 5;

FIG. 6A is an enlargement of a section of the rear portion of the exterior shell as shown in FIG. 6;

FIG. 7 is an exploded view of the dual layer of resilient shock absorbing material of the instant invention;

FIG. 8 is a cross-sectional view through line 8-8 of FIG. 7;

FIG. 9 is a rear view of the walking brace of the invention; and

FIG. 10 is a rear view of the walking brace of the invention with the foam and rear fastening strap removed.

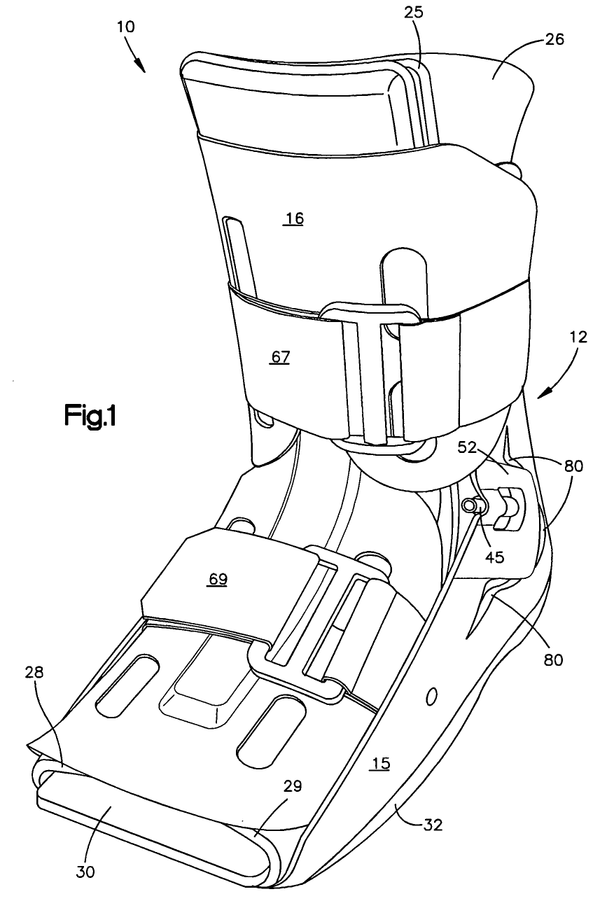

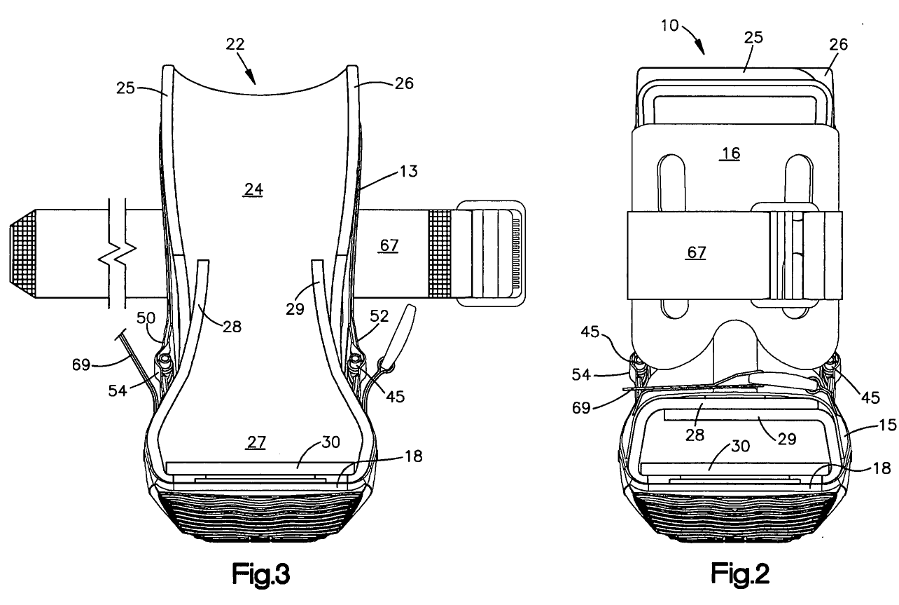

### DETAILED DESCRIPTION OF THE PREFERRED EMBODIMENT

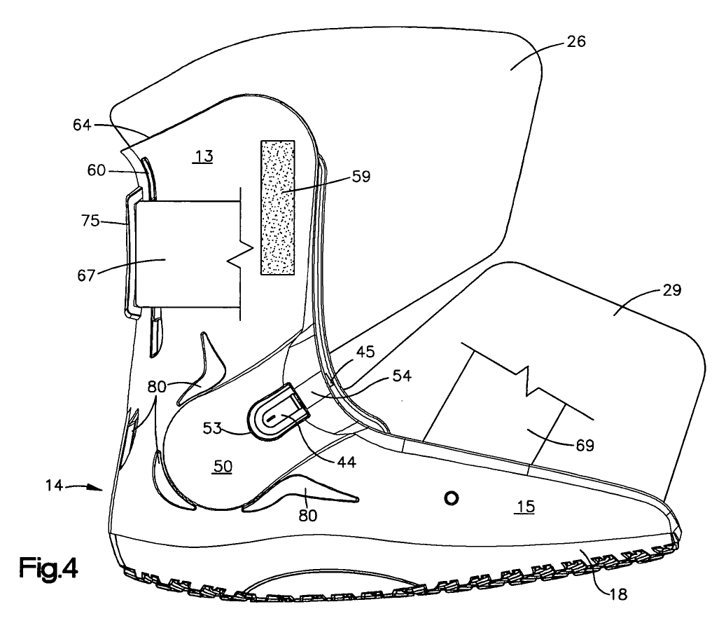

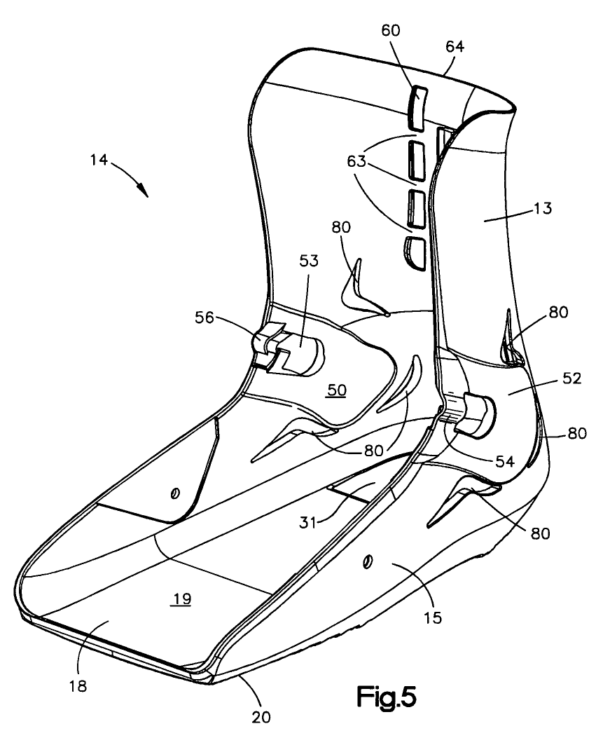

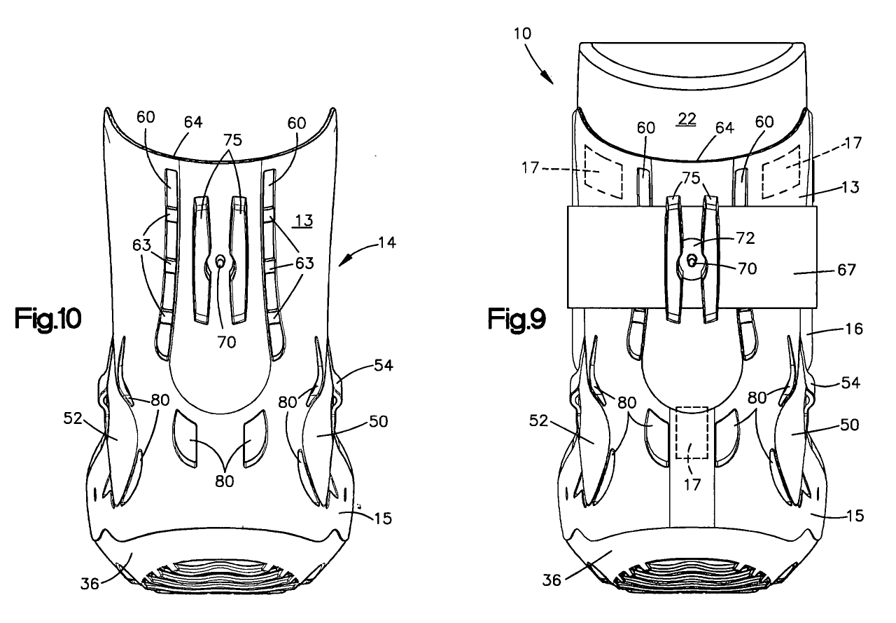

Referring to the figures, a preferred embodiment of a walking brace 10 of the present invention includes a rigid exterior shell 12 comprising a rear portion 14 and a front shell portion 16. Rear portion 14 includes leg portion 13 and foot portion 15. Leg portion 13 of rear portion 14 is adapted to fit around the anterior surface and sides of the patient's lower leg. Disposed below foot portion 15 is sole 18. Foot portion 15 and sole portion 18 are adapted to fit around the sides and bottom of the patient's foot. Disposed along the interior surfaces of rear portion 14 is a foam liner 22, preferably of an open cell foam material. Foam liner comprises a leg portion 24 that includes two forwardly extending flaps 25 and 26 that wrap about the front of the lower leg, and a foot portion 27 that includes two upwardly extending flaps 28 and 29 that wrap generally over the top of the patient's foot. Foam liner 22 can be secured to rear portion 14 of exterior shell 12 by known fastening means. One preferred fastening means is strips of hook material 17 (FIG. 9) adhered to the inner surface of rear portion 14, which hooks releasably engage the outer surface of foam liner 22. The front shell portion 16 and the rear shell portion 14 protect and support the injured leg while the foam liner 22 cushions the leg along the shell portions. The shell portions are held in place on the user's leg by leg portion fastening strap 67 and foot portion fastening strap 69. Front shell portion 16 also can be provided on its inner surface with one or more strips of loop-bearing material (not shown) that engage corresponding strips of hook bearing material 59 (FIG. 4) on rear shell portion 14.

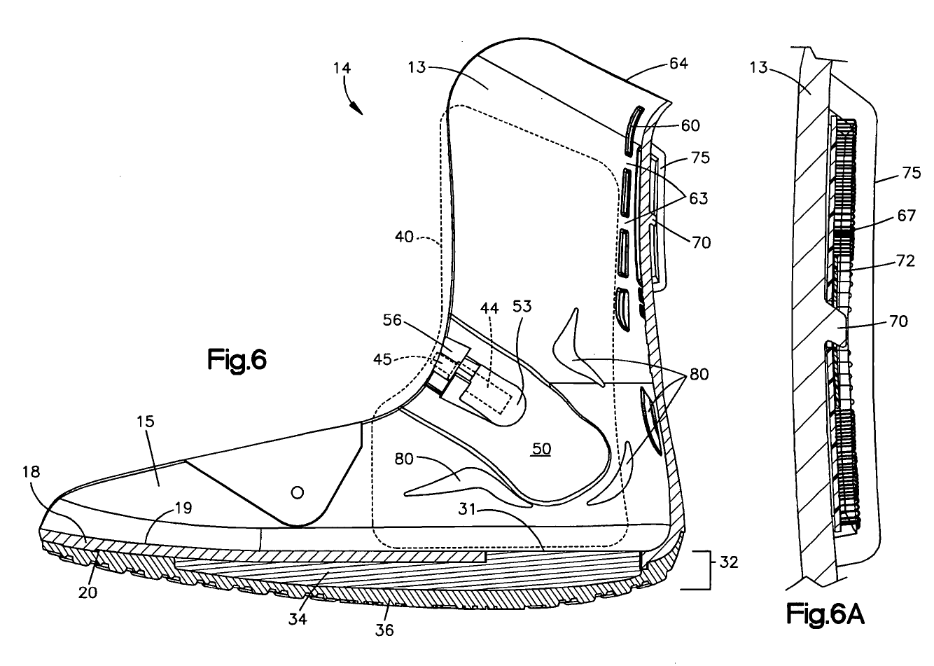

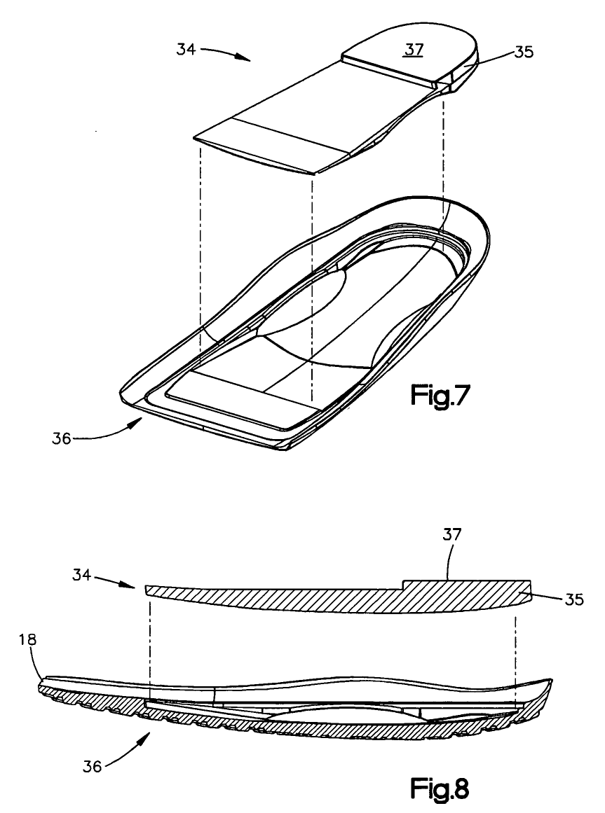

Sole portion 18 includes an upper surface 19 and a lower surface 20. In accordance with the invention, lower surface 20 is provided with a dual layer 32 of a shock resistant material. Referring to FIGS. 6-8, dual layer 32 comprises an upper layer 34 of a relatively softer material and a lower layer 36 of a relatively more durable material. The relatively softer material is lighter weight and provides greater cushioning than the relatively durable material. The lower layer 36 of durable material extends along the bottom of the walking brace and contacts the ground when the patient is walking. The upper layer 34 of softer material 34 is disposed substantially between more durable material 36 and the lower surface 20 of sole portion 18. A particularly suitable material for the softer material 34 is an ethylene vinyl acetate polymer. Suitable materials for more durable layer 36 include polyvinyl chloride and styrene-butadiene rubber, with thermoplastic rubber being particularly preferred.

Sole portion 18 is provided with an opening 31 in the general area of the patient's heel. Opening 31 extends completely through sole portion 18 from lower surface 20 to upper surface 19. Opening 31 can be provided with a circumferential ridge. Upper layer 34 of dual layer 32 includes a heel portion 35 which is of the same shape as opening 31 and is configured to extend upwardly therethrough. Heel portion 35 has an upper surface 37 such that when dual layer 32 is positioned in engagement with sole portion 18, the upper surface 37 of heel portion 35 is generally coplanar with or only slightly above the upper surface 19 of sole portion 18. While heel portion 35 may be manufactured separately and adhered to shock absorbing upper layer 34, it is more expedient from a manufacturing standpoint to provide heel portion 35 and upper layer 34 as a single integral piece. Advantageously, dual layer 32 provides shock absorbency and comfort for the user, yet can be made thinner than prior art single layer shock absorbent members, to allow a lower overall profile of the sole of the walking brace. This means that the injured foot is not raised as high relative to the uninjured foot when the patient is walking, providing greater comfort and mobility for the patient. In a preferred embodiment, the thickness of dual layer 32 can be on the order of about ¾-1 inch, measured form the top surface 37 of heel portion 35, compared to a thickness of about 1¼ for prior art walking braces. Further, the invention is advantageous in that the patient never exerts weight, either directly or indirectly, through the bottom of the patient's heel against the rigid plastic of the sole portion of the exterior shell. The bottom of the patient's heel only engages inner sole liner 30, and indirectly layer 34 of softer material and then layer 36 or more durable material of dual layer 32. The present invention reduces even further the load damage on the patient's heel that can be caused with certain devices of the prior art, which is particularly beneficial to those patients having neuropathy in the foot.

In another aspect of the invention, the interior surfaces of both the rear and front shell portions 14, 16 are preferably provided with a plurality of inflatable air cells that may be actively or passively inflated as taught in the aforementioned prior art patents, U.S. Pat. No. 3,955,565 and U.S. Pat. No. 5,577,998. The walking brace of the invention can also include one or more pre-inflated cells to cushion the leg against the shell portions to provide greater comfort. Other arrangements of air cells and liners may be used without departing from the spirit of the invention disclosed and claimed herein. In particular, medial and lateral air cells can be used to provide pressure on the leg to speed healing and provide greater comfort.

In the illustrated preferred embodiment of the invention, the walking brace comprises a lateral inflatable air cell 40 shown in phantom lines in FIG. 6, and a medial inflatable air cell (not shown), these air cells being disposed along the inner surfaces of the medial and lateral sides, respectively, of rear shell portion 14. The lateral air cell and the medial air cell are gaseously independent, allowing the pressure in each to be adjusted separately. The inflatable air cells can be constructed of two sheets of flexible plastic sealed about their perimeters to make a gas impermeable packet. Each air cell contains therein a resilient passive reinflation means, preferably a piece of foam, as disclosed in prior U.S. Pat. No. 5,577,998. Each air cell has a communication means 44 extending from the air cell to the exterior of the shell 12 and terminating in a closeable air cell port 45 (see FIG. 4). The communication means 44 links the interior of each adjustable air cell with the atmosphere. The communication means 44 is preferably made of flexible plastic tubing. Each air cell port 45 is provided with a closeable sealing means to trap air in the air cell and maintain the air cell at a constant volume. The sealing means can comprise, for example, a hinge stopper or a rotatable valve. When the sealing means is open, the foam piece expands within the air cell causing the air cell to expand by bringing air in from its associated air cell port 45.

In accordance with the invention, rear portion 14 of exterior shell 12 is provided on either side with protruding regions 50, 52 that receive communication means 44 of the lateral and medial inflatable air cells, respectively. The structure of the protruding regions will be described in terms of protruding region 50, it being understood that the structure of protruding region 52 is substantially identical. The wall of protruding region 50 includes an opening 53 for accommodating the end of communication means 44 that connects to the air cell 40, so that the end does not create an undesirable pressure point against the user's leg. Extending forwardly from opening 53 is a channel 54 that guides communication means 44 and associated air cell port 45 toward the front of walking brace 10. Communication means 44 is secured within channel 54 by an internal hook member 56, so that associated air cell port 45 is always readily accessible to the user, yet does not extend so far from the walking brace 10 as to get in the user's way. It will be appreciated that the construction of rear portion 14 of exterior shell 12 facilitates the ready access by the user to the ports 45 of the lateral and medial inflatable air cells, respectively.

In yet another aspect of the invention, rear portion 14 of exterior shell 12 is expandable to accommodate patients whose lower leg has become enlarged or swollen, such as with edema. To accomplish this, the rear wall of leg portion 13 of rear portion 14 is provided with one or more longitudinal slits 60. The slits 60 may extend all the way up to the top edge 64 of leg portion 13, or, as shown in the illustrated embodiment, they may terminate slightly below the top edge 64 of leg portion 13. Longitudinal slits 60 can be provided with one or frangible septa 63 disposed along their length. To expand the width of leg portion 13, if the longitudinal slits 60 extend all the way up to the edge of leg portion 13, then the medial and lateral sides of leg portion 13 can be simply pulled apart from one another. If longitudinal slits do not extend all the way to the top edge of leg portion 13, as shown, then the edges 64 can be cut first to allow the medial and lateral sides of leg portion 13 to be pulled apart from one another. If it is desired to expand the width of leg portion 13 still further, then one or more of frangible septa 63 can be cut, starting from the uppermost septum 63 and continuing down through as many septa as are necessary to be cut to allow expansion of leg portion 13 to accommodate the width of the user's lower leg. If the user's leg is subsequently of a smaller width, such as if the swelling or edema subsides, then the medial and lateral sides of leg portion 13 can be urged back toward their original position and held in place by leg portion fastening strap 67.

In yet another aspect of the invention, it may be desirable from time to time to replace leg portion fastening strap 67. In prior walking braces, the leg portion fastening strap was attached by a rivet or similar permanent fastening means to the rear of leg portion 13. In the illustrated preferred embodiment best shown in FIGS. 6A, 9, and 10, leg portion fastening strap 67 is secured to leg portion 13 by a pin member 70 that is integrally formed on the back facing surface of the rear wall of leg portion 13. A washer 72 is integrally formed with or permanently secured to a surface of strap 67. Retaining means in the form of bars 75 integrally formed on either side of pin member 70 define slots through which the fastening strap 67 is received in sliding engagement. To position strap 67 on rear portion 14 of shell 12, the strap 67 is threaded through the slots of retaining bars 75, and the center of washer 72 is pressed over pin member 70. The retaining bars 75 keep the strap 67 in appropriate position on leg portion 13 and reduce stresses on pin member 70 and washer 72. If strap 67 becomes damaged or otherwise needs to be replaced, then the user can pry the washer 72 of the strap 67 off of the pin member 70, such as with a flathead screwdriver, and slide strap 67 out of the slots. The user can replace a new strap 67 in the slots of retaining bars 75, and position washer 72 of the new strap 67 over pin 70 member. Thus it is not necessary to replace an entire brace when only a strap needs to be replaced.

In still another aspect of the invention, rear portion 14 of exterior shell is provided with a plurality of holes 80, that serve to improve ventilation at the skin of the injured leg. The ventilation holes 80 also reduce the overall weight of the brace 10, which further adds to the comfort of the user.

While the invention has been shown and described with respect to a particular embodiment, this is for purposes of illustration rather than limitation. The inventor envisions, and it will be apparent to those skilled in the art, that other variations and modifications of the embodiment shown and described herein are all within the intended spirit and scope of the invention. Accordingly, the patent is not to be limited in scope and effect to the specific embodiment shown and described nor in any other way that is inconsistent with the extent to which the progress of the art has been advanced by the invention.

1. A walking brace for the lower leg of a patient, said walking brace comprising:
a leg portion adapted to fit substantially around the lower leg of a patient,
a rigid sole portion adapted to fit beneath the foot of a patient, said rigid sole portion having an opening therethrough in the area of the patient's heel, said opening extending through said upper surface and said lower surface of said rigid sole portion, such that no portion of said rigid sole portion is disposed below the patient's heel; and
a dual layer of resilient shock-absorbing material disposed along said bottom surface of said rigid sole portion, said dual layer of resilient shock-absorbing material having a heel portion extending upwardly through said opening in said sole portion to a height no greater than slightly above said upper surface of said rigid sole portion, said dual layer comprising an upper layer of a relatively softer material and a lower layer of a relatively more durable material, said upper layer being in contact with the patient's heel, said lower layer being in contact with the ground when the patient walks.

2. The walking brace of claim 1 wherein said heel portion is integrally formed with said upper layer of said dual layer of shock absorbing material.

3. The walking brace of claim 1 wherein said heel portion has an upper surface substantially coplanar with said upper surface of said sole portion.

4. The walking brace of claim 1 wherein the material of the lower layer of said dual layer has a greater density than the material of said upper layer of said dual layer.

5. The walking brace of claim 4 wherein said lower layer of said dual layer is made of a material selected from the group consisting of thermoplastic rubber, polyvinyl chloride, and styrene-butadiene rubber.

6. The walking brace of claim 5 wherein said upper layer is made of an ethylene vinyl acetate polymer.

7. The walking brace of claim 1 wherein the thickness of said dual layer from the top surface of the heel portion to the bottom surface of the bottom layer is no greater than about 1 inch.

8. The walking brace of claim 1 further comprising a foam layer disposed over the upper surface of said sole portion.

9. The walking brace of claim 1 wherein said leg portion comprises a rigid shell integral with said sole portion.

10. The walking brace of claim 9 further comprising at least one inflatable air cell for providing therapeutic pressure to said leg.

11. The walking brace of claim 10 wherein said at least one inflatable air cell comprises a communication means by which the interior of said air cell can be in fluid communication with the atmosphere.

12. The walking brace of claim 11 wherein said rigid shell comprises at least one protruding region for receiving said communication means, such that said communication means is directed toward the front side of said walking brace.

13. The walking brace of claim 9 wherein said rigid shell comprises a rear shell member and a front shell member, said rear shell member comprising means for expanding the width thereof.

14. The walking brace of claim 13 wherein said expansion means comprises at least one longitudinal slit, such that the portions of said rear shell member on either side of said longitudinal slit can be pulled apart, such that said rear shell member can expand laterally.

15. The walking brace of claim 13 wherein the upper end of said longitudinal slit terminates at the upper edge of said rear shell member.

16. The walking brace of claim 13 wherein the upper end of said longitudinal slit terminates below the upper edge of said rear shell member, and the portion of the shell member between the upper edge of the shell member and the upper end of the longitudinal slit is cut to allow the portions of the shell portion on either side of the slit can be pulled apart to allow the width of the rear shell member to expand.

17. The walking brace of claim 14 wherein said expansion means comprises a plurality of longitudinally aligned slits, said longitudinally aligned slits being separated one from the other by one or more frangible septa.

18. The walking brace of claim 14 wherein said expansion means comprises a plurality of longitudinal slits spaced laterally from one another.

19. A walking brace for the lower leg of a patient, said walking brace comprising:
a leg portion adapted to fit substantially around the lower leg of a patient, and
a rigid sole portion adapted to fit beneath the foot of a patient,
said leg portion comprising a rigid shell integral with said sole portion, said rigid shell comprising a rear shell member and a front shell member, said rear shell member comprising means for expanding the width thereof.

20. The walking brace of claim 19 wherein said expansion means comprises at least one longitudinal slit, such that the portions of said rear shell member on either side of said longitudinal slit can be pulled apart to expand said rear shell member laterally.

21. The walking brace of claim 20 wherein the upper end of said longitudinal slit terminates at the upper edge of said rear shell member.

22. The walking brace of claim 20 wherein the upper end of said longitudinal slit terminates below the upper edge of said rear shell member, and the portion of the shell member between the upper edge of the shell member and the upper end of the longitudinal slit is cut to allow the portions of the shell portion on either side of the slit to be pulled apart to expand the width of the rear shell member.

23. The walking brace of claim 19 wherein said expansion means comprises a plurality of longitudinally aligned slits, said longitudinally aligned slits being separated one from the other by one or more frangible septa.

24. The walking brace of claim 19 wherein said expansion means comprises a plurality of longitudinal slits spaced laterally from one another.

25. The walking brace of claim 19 wherein said expansion means comprises at least one longitudinal slit having two ends, a least one of said ends terminating at a frangible septum, such that when said septum is broken said rear shell member can expand laterally.

26. The walking brace of claim 19 further comprising at least one adjustable air cell having a means for communication between the interior of said air cell and the atmosphere, wherein said rigid shell comprises at least one protruding region for receiving said air cell communication means, said region constructed such that said communication means is directed toward the front side of said walking brace.

27. The walking brace of claim 19 wherein said rigid sole portion has an opening therethrough in the area of the patient's heel, said opening extending through said upper surface and said lower surface of said rigid sole portion, such that no portion of said rigid sole portion is disposed below the patient's heel.

28. The walking brace of claim 27 further comprising a dual layer of resilient shock-absorbing material disposed along said bottom surface of said rigid sole portion, said layer of resilient shock-absorbing material having a heel portion extending upwardly through said opening in said sole portion to a height no greater than slightly above said upper surface of said rigid sole portion, said dual layer comprising an upper layer of a relatively softer material and a lower layer of a relatively more durable material, said upper layer being in contact with the patient's heel, said lower layer being in contact with the ground when the patient walks.

29. A walking brace for the lower leg of a patient, said walking brace comprising:
a leg portion adapted to fit substantially around the lower leg of a patient, and
a rigid sole portion adapted to fit beneath the foot of a patient,
said leg portion comprising a rigid shell integral with said sole portion, and
at least one adjustable air cell having a means for communication between the interior of said air cell and the atmosphere, wherein said rigid shell comprises at least one protruding region for receiving said air cell communication means, said region constructed such that said communication means is directed toward the front side of said walking brace.

30. A walking brace for the lower leg of a patient, said walking brace comprising:
a leg portion adapted to fit substantially around the lower leg of a patient,
a rigid sole portion adapted to fit beneath the foot of a patient, and
a strap for securing the leg portion about the lower leg of the patient, said strap being removably secured to said leg portion.

31. The walking brace of claim 30 wherein said leg portion comprises a pin member protruding from an outer surface thereof and retaining means for slidably receiving said strap, and said strap comprises a means releasably engageable with said pin member, such that strap member can be released from said pin member and slidably removed from said retaining means.
